(19) United States
(12) Patent Application Publication
(10) Pub. No.: US 2024/0285938 A1
(43) Pub. Date: Aug. 29, 2024

(54) ANCHORED ELECTRODE SYSTEMS FOR LONG-TERM NEUROSTIMULATION

(71) Applicants: FUNDACIO INSTITUT CATALA DE NANOCIENCA I NANOTECHNOLOGIA (ICN2), Cerdanyola del Valles (ES); INSTITUCIÓ CATALANA DE RECERCA I ESTUDIS AVANÇATS, Barcelona (ES); CONSORCIO CENTRO DE INVESTIGACIÓN BIOMÉDICA EN RED (CIBER), Madrid (ES)

(72) Inventors: Steven T. WALTSON, Los Angeles, CA (US); José A. GARRIDO, Sant Just Desvern, Barcelona (ES); Damiá VIANA, Barcelona (ES); Elisabeth PRATS ALFONSO, Barcelona (ES); Xavier Illa VILA, Artés (ES)

(21) Appl. No.: 18/569,158

(22) PCT Filed: Nov. 24, 2021
(86) PCT No.: PCT/EP2021/082725

§ 371 Date: Dec. 11, 2023

(30) Foreign Application Priority Data
Jun. 11, 2021 (EP) 21382525.0

(51) Int. Cl. A61N 1/05

(52) U.S. Cl. CPC A61N 1/0539; A61N 1/0531; A61N 1/0534

(57) ABSTRACT

The present invention relates to an electrode comprising an electrode base material, a carbon material, and an anchoring layer, wherein the anchoring layer is located between the electrode base material and the carbon material and comprises an anchoring structure having a chain of at least eight consecutive covalently bonded atoms.

### TECHNICAL FIELD

The project leading to the present application has received funding from the Graphene Flagship programme (Graphene Core2) under the European Union's Horizon 2020 research and innovation programme (grant agreement No 785219).

### BACKGROUND AND SUMMARY

The present invention provides an electrode, preferably an electrode for neurostimulation, e.g. for cortical and/or deep brain stimulation. The invention also provides a method for preparing the electrode. The invention further provides an electrode carrier for detecting, receiving and/or inducing physiological electrical signals, comprising the electrode. In addition, the invention relates to the use of a specific anchoring material for attaching a carbon material to an electrode base material.

The present invention relates to the technical field of neurostimulation, in particular to cortical and/or deep brain stimulation. Cortical and/or deep brain Stimulation are methods for the diagnosis and therapy of neurodegenerative diseases such as inter alia the Parkinson's disease, epilepsy, and chronic pain. Electrical stimulation by means of leads which are implanted into brain areas or regions like the subthalamic nucleus and/or the globus pallidus internus can alleviate symptoms, such as tremor symptoms of a patient suffering from a drug-resistant Parkinson's disease. Further, the signals from a brain region or area at which the leads were implanted can be recorded and the state of the brain tissue can be determined using impedance measurements.

In this relation, neuroprosthetic devices are powerful tools to monitor, prevent and treat neural diseases, disorders and conditions by interfacing electrically with the nervous system. They are capable of recording and stimulating electrically neural activity once implanted in the nervous tissue. Currently, most neuroprosthetic technologies apply electrodes interfacing with neural tissue.

Standard commercially available neural interfaces are based on metallic electrodes made of platinum Pt, platinum-iridium (Pt/Ir), iridium oxide (IrOx) or titanium nitride (TiN). Those materials interact with the living tissue through a combination of Faradaic and capacitive currents, offer a limited chemical stability and are rigid. Metals' performance strongly drops in microelectrodes of tens of micrometres in diameter. Further, metals degrade over continuous tissue stimulation.

In order to avoid some of these disadvantages, electrodes with a coating applied to an electrode base material were suggested. However, such coatings bring along other disadvantages which include chemical and mechanical degradation of the coatings when the electrode is in operation.

In this regard graphene based (coating) materials are very promising. This is not only because these materials are highly inert and mechanically robust and therefore avoid degradation problems. Graphene based materials are highly favorable also because of their electrical properties and flexibility. Graphene based materials thus appear ideal for safe electrical interfacing in aqueous environments.

Nevertheless, also for these graphene based (coating) materials, further improvement is desired, in particular an improved adhesion of films based on graphene oxide to metallic electrode base materials.

Immobilization of graphene oxide has been described in connection with molecular based electronics (K. Yu et al., Chemistry Letters 2019, Vol. 48, No. 9, page 1101 to 1104). K. Yu et al. immobilized graphene oxide monolayers on a single crystal phosphorus-doped Si(111) wafer (with H—Si surface) by applying 4-vinylaniline molecules as intermediate linkers. The specific chemistry applied in this work involved visible-light irradiation of the wafer in contact with 10 mM deoxygenated 4-vinylaniline/decane solution in order to form covalent bonds from vinyl carbon atoms of 4-vinylaniline to silicon atoms of the wafer. The authors emphasize that 4-vinylaniline was not an arbitrary choice for immobilizing graphene oxide on the wafer. Three reasons are provided for choosing this linker: First, the 4-vinylaniline molecule contains a conjugated structure (aromatic moiety), which enhances the electrical conductivity between the base electrode and the upper functionalities. Second, the vinyl group of the 4-vinylaniline molecule has a strong affinity to the covalent couple on the H—Si surface through the robust Si—C bond and can be expected to form self-assembled monolayers. Third, it has been reported that the amino group can be reacted with some types of oxygen functional groups and graphene oxide immobilized onto the —NH₃ terminated substrate.

Focusing on treatments for central nervous system injuries and subsequent rehabilitation, Fu et al. studied electrical stimulation combined with graphene-oxide-based membranes on neural stem cell proliferation and differentiation (Artificial Cells, Nanomedicine, and Biotechnology 2019, Vol. 47, No. 1, page 1867-1876). The graphene-oxide-based membrane used was a conductive poly(L-lactic-co-glycolic acid)/graphene oxide composite membrane and it was prepared by mixing graphene oxide and poly(L-lactic-co-glycolic acid) using the solution evaporation method.

The preparation method and membranes described by Fu et al. have certain disadvantages. The membranes' long-term stability is uncertain. The poly(L-lactic-co-glycolic acid) used is well-known for its biodegradability and the rate of biodegradation might depend on factors that are difficult to control, such as the crystallinity of the polymer in a specific membrane or in part of a membrane. In addition, the membrane thickness is difficult to control and parts of the membranes may be poor in graphene oxide and rich in poly(L-lactic-co-glycolic acid) or vice versa. Stimulation might thus be inconsistent at different membrane locations. At specific locations, detection of neural signaling might even fail.

It is an object of the present invention to provide an electrode that can be produced at low effort within tight manufacturing tolerances, which enables a reliable neurostimulation and which possesses stability even when exposed to surrounding tissue for a long time.

The aforesaid object is achieved by the subject-matter of the independent claims. Advantageous configurations of the invention are described in dependent claims.

According to the present invention, an electrode comprising an electrode base material, a carbon material, and an anchoring layer is provided, wherein the anchoring layer is located between the electrode base material and the carbon material and comprises an anchoring structure having a chain of at least eight consecutive covalently bonded atoms.

The basic idea underlying the present invention is increasing the mechanical stability and the safety of electrodes for neurostimulation, thereby increasing the acceptance of neurostimulation technology even for long term applications in humans. The invention avoids delamination of coatings from electrode base material as the carbon material is anchored by the anchoring structure in the anchoring layer.

The electrode according to the present invention is preferably an electrode for neurostimulation, in particular an electrode for cortical and/or deep brain stimulation. The word “electrode” as used herein is not limited to a specific shape. It refers to any object that can be implanted into a human tissue and that is capable of electrical interaction with adjacent neuronal tissue. Electrical interaction may include a sensing of a (physiological) electrical signal in adjacent neuronal tissue and/or a transmission of an external electrical stimulus into such tissue. A transmitting device, e.g. an electrically conductive wire, can be connected to the electrode, for further transmission of the (physiological) electrical signal and/or for transmitting the external electrical stimulus to the electrode.

According to the invention, the electrode base material can be any material which is suitable as an electrode base material, e.g., any material capable of transmitting the external electrical stimulus towards the neuronal tissue and/or capable of transmitting the (physiological) electrical signal towards the transmitting device and which can be equipped with the anchoring layer of the invention. A preferred electrode base material comprises gold, iridium, platinum, titanium, silver, and/or palladium. A particularly preferred electrode base material comprises platinum Pt, platinum-iridium (Pt/Ir), iridium oxide (IrOx) or titanium nitride (TiN).

The electrode of the invention comprises a carbon material. The carbon material is not limited to a specific carbon material. Any carbon material that can be anchored as described herein and that has desired properties, i. e. with regard to electrical conductivity, flexibility, mechanical robustness, and inertness when exposed to neuronal tissue, is suitable in the context of the invention.

A preferred carbon material is a graphene material. Graphene materials include graphene, graphene oxide (GO), reduced graphene oxide (rGO) and other kinds of modified graphene. This allows for the carbon material to be present in the form of extremely thin carbon material layers. As will be understood by the following description, anchoring ensures that graphene, e.g. GO or rGO single layers will preferably be lying on the surface of the anchoring layer and be fixed in this orientation by the anchoring structure.

Graphene is an allotrope of carbon that is consisting of single layers of atoms arranged in a two-dimensional honeycomb lattice. In practice, this ideal form of graphene is not reached as separation into single layers is not complete and functional groups, in particular oxygen functional groups are present at least at the edges of the single layers.

GO can be obtained by treating graphite with strong oxidizers. GO includes oxygen functional groups also at carbon atoms within the single layer. From M. Chhowalla et al., “Chemically Derived Graphen Oxide: Towards Large-Area Thin-Film Electronics and Optoelectronics”, Advanced Materials, 2010, Vol. 22, page 2393 to 2415, it is known that chemically derived graphene possesses a unique set of properties arising from oxygen functional groups that are introduced during chemical exfoliation of graphite.

rGO can be obtained from GO by partial reduction which reduces the GO's original content of oxygen functional groups.

Most preferably, the carbon material is a GO or a rGO. As compared to graphene, a higher content of oxygen functional groups enables an even more reliable anchoring of GO and rGO.

According to the invention, an anchoring layer is located between the electrode base material and the carbon material. Preferably, a distal surface of the anchoring layer is in contact with the carbon material and a proximal surface of the anchoring layer is in contact with the electrode base material.

The word “distal” is used herein when referring to a feature that is (or will be) further away from the electrode base material than a corresponding “proximal” feature.

The invention does not exclude further materials or layers. In particular, at least one additional layer may be present. The additional layer may be present, for example, between the electrode base material and the anchoring layer, or between the anchoring layer and the carbon material.

According to the invention, the anchoring layer comprises an anchoring structure having a chain of at least eight consecutive covalently bonded atoms. Hydrogen atoms are ignored when counting the number of consecutive covalently bonded atoms. The anchoring structure thus has a chain of at least eight consecutive covalently bonded atoms other than hydrogen atoms.

Typical anchoring structures of the invention have much larger chain lengths. A preferred anchoring structure has a chain of at least 10, preferably of at least 12, more preferably of at least 14, in particular of at least 16, e.g. of at least 20 consecutive covalently bonded atoms, in each case of atoms other than hydrogen atoms.

The anchoring structure may comprise a polyalkylene oxide chain, e.g. a polyethylene oxide chain. Preferably, the polyalkylene oxide, e.g. the polyethylene oxide chain, has one of the above-indicated minimum chain lengths of consecutive covalently bonded atoms.

Preferably, at least part of the anchoring layer is a monolayer. A monolayer is a layer which has a thickness defined by the length of the molecules from which it is made. Anchoring layer that are monolayers can be prepared very easily by making the anchoring layer from molecules that tend to form self-assembled monolayers on surfaces. The inventors have found that polyalkylene oxides and, in particular polyethylene oxides, tend to form such self-assembling monolayers on common electrode base materials.

According to the invention, it is further preferred when a distal end of the anchoring structure is covalently bound to the carbon material. This ensures an even more stable binding of the carbon material to the anchoring layer and thus an improved structural integrity of the electrode. Alternatively or in addition, distal ends of anchoring structures can carry an ionic charge, e.g. an ammonium residue, that can interact with an ionic charge, e.g. a carboxylic group, of the carbon material, e.g. of a graphene oxide. Although covalent bonds are preferred, ionic interactions may be well sufficient. This holds true in particular when the density of charges at the surface of the graphene oxide and of opposed charges at the distal surface of the anchoring layer are both high.

Preferably, the distal end of the anchoring structure is covalently bound to the carbon material by a distal substructure A_{d}-X (with single bond from A_{d} to X) or A_{d}=X (with a double bond from A_{d} to X) wherein
A_{d} is a distal hetero atom, preferably a distal nitrogen atom, of the anchoring structure, and
X is an atom, preferably a carbon atom, of the carbon material.

Such covalent bonds can be formed very easily, when distal amino groups of the anchoring layer react with typical functional groups at the surface of carbon materials (i.e. with epoxy groups, carboxyl groups, aldehyde groups). It is apparent for a skilled person that amino nitrogen is not the sole nucleophile that can be provided at the surface of the anchoring layer and that other functional groups at the surface of carbon materials may be involved in the formation of covalent bonds. The invention is thus not limited to single or double bonds from nitrogen to carbon.

An efficient formation of covalent bonds can be expected when anchoring layers with strong nucleophiles, i.e. hydrazides, at their distal surfaces are converted with carbon materials such as graphene oxide. This leads to specific distal substructures. Most preferably, the distal end of the anchoring structure is thus covalently bound to the carbon material by a distal substructure C(O)—NH—NH—X (with single bond to X) or C(O)—NH—N═X (with double bond to X) wherein
C(O)—NH—NH or C(O)—NH—N is a subunit of the anchoring structure, and
X is an atom, preferably a carbon atom, of the carbon material.

Preferably a proximal end of an anchoring structure is coupled to the electrode base material. Most preferably a distal end of one anchoring structure is covalently bound to the carbon material and a proximal end of the same anchoring structure is coupled to the electrode base material. This leads to a further increase of the structural integrity of the electrode of the invention in which a delamination of the covalently bound carbon is almost impossible.

Preferably, the gold, iridium, platinum, titanium, silver, and/or palladium (as already mentioned hereinabove as a possible constituent of the electrode base material) is at a surface of the electrode base material. Each of these metals allow for a coupling of an anchoring structure by applying well-known chemistry for coupling to or even forming covalent bonds to the specific metal.

Attachment to platinum is described, for example, by Li, Z., Chang, S. and Williams, R., 2003, Langmuir, 19(17), pp. 6744-6749. Attachment to Iridium is described, for example, by Yee, C., Jordan, R., Ulman, A., White, H., King, A., Rafailovich, M. and Sokolov, J., 1999, Langmuir, 15(10), pp. 3486-3491. Attachment to Palladium is described, for example, by Love, J., Wolfe, D., Haasch, R., Chabinyc, M., Paul, K., Whitesides, G. and Nuzzo, R., 2003, Journal of the American Chemical Society, 125(9), pp. 2597-2609. Attachment to Titanium is described, for example, by Mani, G., Johnson, D., Marton, D., Dougherty, V., Feldman, M., Patel, D., Ayon, A. and Agrawal, C., 2008, Langmuir, 24(13), pp. 6774-6784. Attachment to silver is described, for example, by Stewart, A., Zheng, S., McCourt, M. and Bell, S., 2012, ACS Nano, 6(5), pp. 3718-3726. It is generally assumed that this well-known chemistry for coupling to metal surfaces would typically result in covalent bonds. Recent results have demonstrated that this is not necessarily the case (M. S. Inkpen et al., 2019, Nature Chemistry, Vol. 11, pp. 351-358). Herein, this is taken into account by using the more general terms “coupling” or “coupled”, rather than “covalent binding” or “covalent bond” in connection with the attachment of the anchoring structure to the electrode base material.

Preferably, a proximal end of the anchoring structure is coupled to the electrode base material by a proximal substructure Aₚ-Y wherein
Aₚ is a proximal hetero atom, preferably a proximal sulfur atom, of the anchoring structure,
Y is an atom, preferably a gold atom, of the electrode base material, and
   between Aₚ and Y is a coupling, e.g. a covalent bond, of Aₚ to Y.

The invention further provides a method for preparing an electrode, which comprises the following steps:
a) contacting an electrode base material with an anchoring composition which comprises an anchoring compound having a chain of at least eight consecutive covalently bonded atoms,
b) contacting the electrode base material obtained from step a) with a carbon material.

There may be one or more optional additional steps between step a) and b), e.g. purification and/or drying steps. The reference to a material “obtained from step a)” means that step a) precedes step b). The electrode base material obtained from step a) may thus be directly obtained from step a) or indirectly obtained from step a) via at least one intermediated step.

A preferred anchoring compound comprises proximal hetero atom Aₚ and distal hetero atom A_{d} and a chain which links Aₚ to A_{d} by at least six consecutive covalently bonded atoms. A particularly preferred anchoring compound comprises proximal hetero atom Aₚ and distal hydrazide substructure C(O)—NH—NH₂ and a chain which links Aₚ to the carbonyl carbon atom of the hydrazide substructure by at least four consecutive covalently bonded atoms.

A preferred chain which links Aₚ to A_{d} or Aₚ to the carbonyl carbon atom of the hydrazide substructure comprises a polyalkylene oxide chain, e.g. a polyethylene oxide chain. Preferably, the polyalkylene oxide chain, e.g. the polyethylene oxide chain, has one of the above-indicated minimum chain lengths of consecutive covalently bonded atoms.

The anchoring compound preferably has a weight average molar weight in a range from 100 to 50.000 Da, e.g. from 200 to 20.000 Da.

A specific example of an anchoring compound is a substituted polyalkylene oxide having a thiol group at one end and a hydrazide group at the other end of a polyethylene oxide chain which links the sulfur atom of the thiol group to the carbonyl carbon atom of the hydrazide group. On one end of the anchoring compound, the hydrazide group can form a covalent bond to a functional group of the GO and on the other end of the anchoring compound, the sulfur atom of the thiol group can bind to the metallic electrode base material, e.g., to gold atoms of the metallic electrode base material.

The anchoring compounds and in particular the substituted polyalkylene oxides were found to self-assemble into monolayers. These monolayers ensure a well-defined but very small distance from the electrode base material to the anchored carbon material. This ensures small resistivity between the substrate material and the graphene layer. A smaller resistivity is favorable as it was found to result in a higher electrode sensitivity and thus provides electrodes capable of sensing even weaker neuronal signals.

Step a) and b) are each preferably carried out under conditions that accelerate the chemical reactions resulting in covalent bonds to atoms of the electrode base material and to carbon atoms of the carbon material, respectively. This may involve, for example, use of specific solvents, application of heat or addition of catalysts in order to further accelerate the formation of covalent bonds.

The invention further provides an electrode obtainable according to the method of the invention.

In addition, the invention relates to an electrode carrier for detecting, receiving and/or inducing physiological electrical signals, wherein the electrode carrier comprises an electrode according to the invention. Preferably, the electrode carrier comprises et least two (e.g. a plurality of) electrodes according to the invention. Most preferably, the at least two (e.g. the plurality of) electrodes define at least two (e.g. the plurality of) electrode areas that are arranged in a zone which is located at an end of the electrode carrier and the electrode areas are spaced apart from one another in the zone.

The physiological electrical signals that can be detected, received and/or induced are preferably electrical signals in brain tissue, most preferably in single neural cells.

The electrode carrier may further comprise a transmitting device, e.g. an electrically conductive wire, connected to or connectable to the electrode(s).

The invention further relates to the use of an anchoring compound having a chain of at least eight consecutive covalently bonded atoms for attaching a carbon material to an electrode base material. The anchoring compound is as defined with regard to the method of the invention.

Features describe in connection with one specific embodiments of the invention, i.e. with the electrode, the electronic device, the method or the use of the invention, do also apply to the other embodiments, if not explicitly stated otherwise.

The invention is described in more detail by the figures and the following non-limiting examples.

### BRIEF DESCRIPTION OF THE FIGURES

It is shown in

FIG. 1 an illustration of an electrode carrier according to an embodiment of the invention, which comprises a plurality of electrodes;

FIG. 2 the layering structure of an electrode according to an embodiment of the invention; and

FIG. 3 a schematic representation of a method according to an embodiment of the invention.

### DETAILED DESCRIPTION

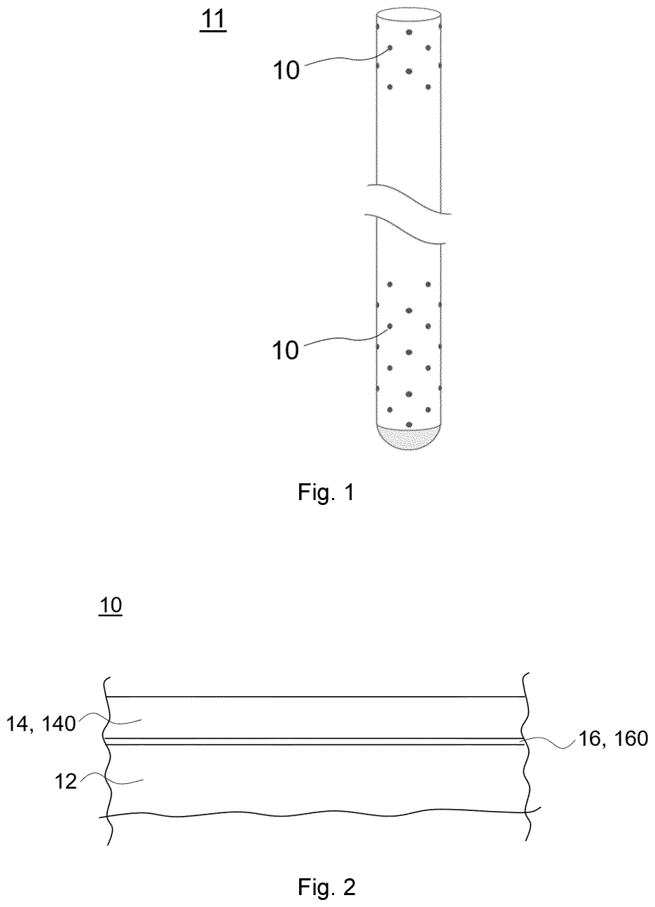

FIG. 1 shows an electrode carrier 11 according to the invention, for detecting, receiving and/or inducing physiological electrical signals.

The electrode carrier 11 comprises a plurality of electrodes 10 according to the invention.

The electrodes 10 define a plurality of electrode areas.

Each electrode area is a spot in FIG. 1.

The lowest part of FIG. 1 shows an end of the electrode carrier 11 and a subgroup of the electrode areas is arranged in a zone which is located at the end of the electrode carrier.

FIG. 1 further shows that the electrode areas are spaced apart from one another in the zone.

The electrode carrier exemplified here further comprises a plurality of electrode areas in another zone which is further away from the end of the electrode carrier and which is shown in the upper part of FIG. 1.

FIG. 2 is a very schematic illustration of the layering structure of an electrode 10 of the electrode carrier 11.

Electrode 10 comprises an electrode base material 12, a carbon material 14 which is in this specific example a graphene material 140 selected from graphene oxide and reduced graphene oxide, and an anchoring layer 16.

As shown in FIG. 2, anchoring layer 16 is located between the electrode base material 12 and the carbon material 14.

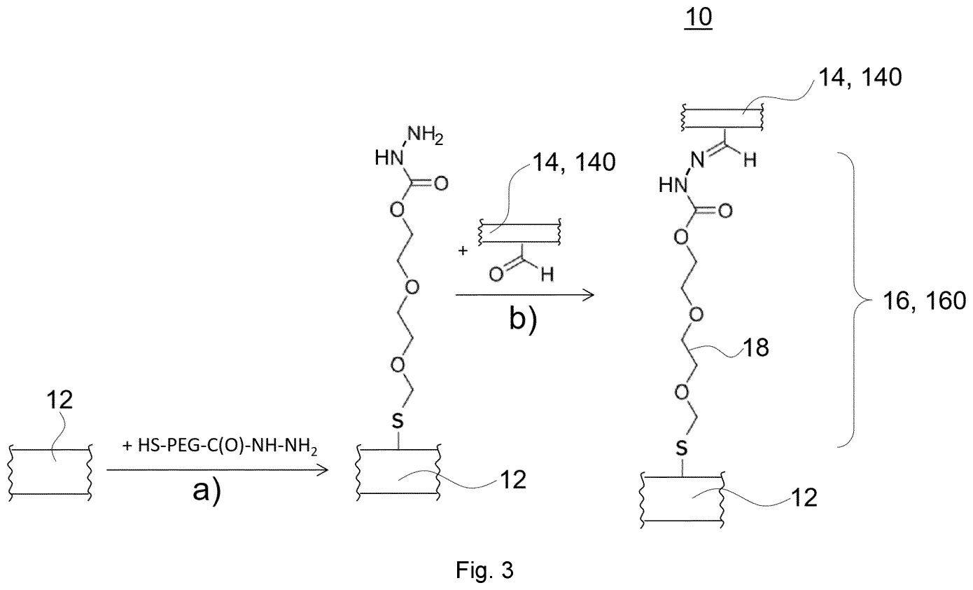

As described in further detail with reference to FIG. 3, anchoring layer 16 is a monolayer 160 which comprises an anchoring structure 18 having a chain of at least eight or even much more (e.g. at least thirty) consecutive covalently bonded atoms.

The method illustrated in FIG. 3 is a method according to the invention for preparing electrode 10.

It comprises step a) of contacting an electrode base material 12 having gold (not shown) at one of its surfaces with an anchoring composition.

The anchoring composition comprises HS-PEG-C(O)—NH—NH2 which is a specific example of an anchoring compound. As PEG in HS-PEG-C(O)—NH—NH2 represents a polyethylene glycol substructure, the HS-PEG-C(O)—NH—NH2 anchoring compound comprises a chain of at least eight consecutive covalently bonded atoms.

The illustrated method further comprises step b) of contacting the electrode base material obtained from step a) with a carbon material 14.

The carbon material 14 in this specific example is a graphene oxide or a reduced graphene oxide, i.e. a specific form of graphene material 140.

Carbon material 14 has functional groups at its surface.

One exemplary aldehyde group is shown here.

Other functional groups are typically present on carbon material surfaces in addition to aldehyde groups.

It is immediately apparent that the size of the anchoring structure 18 in FIG. 3 is much larger than in reality.

FIG. 3 further shows only sections of schematically represented electrode base material 12 and carbon material 14.

Under typical experimental conditions of strep a), an anchoring compound monolayer is expected to form.

This results in an anchoring layer 16 wherein each anchoring structure 18 is surrounded by neighboring anchoring structures 18.

As shown in FIG. 3 on the right, a distal end of the anchoring structure 18 is covalently bound to the carbon material 14 by a distal substructure A_{d}=X wherein A_{d} is a distal nitrogen atom, of the anchoring structure, and X is a carbon atom, of the carbon material.

In the specific example of FIG. 3, the distal substructure is C(O)—NH—N═C, wherein C(O)—NH—N is a subunit of the anchoring structure, and the additional C atom is from the carbon material.

As further shown in FIG. 3 on the right, a proximal end of the anchoring structure 18 is covalently bound to the electrode base material 12.

In the example of FIG. 3, the electrode base material 12 comprises gold (not indicated in the Figure) and the sulfur atom at the proximal end of the anchoring structure 18 is covalently bound to a gold atom at a surface of the electrode base material 12.

More generally, the proximal end of the anchoring structure 18 can be covalently bound to the electrode base material 12 by a proximal substructure Ap-Y wherein Ap is a proximal hetero atom of the anchoring structure, and Y is an atom of the electrode base material 12.

A skilled person is well familiar with the chemistry of metal surface coating and the preference of specific metals (Y) for specific hetero atoms (Ap).

It is self-evident that (by far) not every anchoring compound molecule comprised by an anchoring layer 16 or monolayer 160 would be covalently bound to electrode base material 12 and carbon material 14.

The formation of covalent bonds to these materials depends on many different conditions, including the reactivity of the functional groups at both ends of the anchoring compound molecules, the type of and density of functional groups provided by the carbon material, and the chemical element, e.g. gold, iridium, platinum, titanium, silver, and/or palladium, at the surface of the electrode base material.

The fact that only a portion of the anchoring compound molecules will ever form bonds or, in particular, covalent bonds to the electrode base material or carbon material, is taken into account herein by requiring that the anchoring layer comprises an anchoring structure involved in bonding or covalent bonding to electrode base material and/or carbon material. The word comprises in this context means that the anchoring layer typically comprises further molecules and in particular further anchoring compound molecules that are not involved in covalent bonding.

### Example 1: Anchoring of GO on an Electrode Base Material by an Anchoring Layer

1. A substrate coated with gold was provided. The substrate was cleaned by reactive ion etching.
2. The cleaned substrate was placed in a beaker containing an anchoring composition and the beaker was sealed with Parafilm®. The anchoring composition was composed of 4 mg of Hydrazide-PEGx-Thiol (MW: 2000 Da P/N: AWK1T1, Interchim Uptima) dissolved in 12 mL of ethanol. Hydrazide-PEGx-Thiol is HS-PEG-C(O)—NH—NH2 of FIG. 3.
3. After 12 hours of incubation, the substrate coated with the anchoring layer was washed for two minutes in an ethanol bath and blown dry with nitrogen gas.
4. Next, a GO film was transferred onto the substrate using a wet transfer method (using DI water) and then dried on the hotplate at 50° C. for 10 minutes in order to accelerate the formation of oxygen functional groups of GO with covalent bonds of distal hydrazide residues of the anchoring layer.

The GO film used in step 4 can be prepared as follows:
a) a graphene oxide suspension in water is filtered through a porous membrane thereby forming a GO structure on the membrane top. Suitable GO suspensions contain from 0.001 to 5 mg of GO per milliliter and the volume of suspension can be chosen in a range from 5 to 1000 mL;
b) the GO structure is transferred from the membrane onto a sacrificial substrate, whereby the GO structure is placed between the membrane at the top and the sacrificial substrate at the bottom;
c) the membrane is removed from the top, whereby the GO structure remains attached onto the sacrificial substrate, resulting in a GO film on sacrificial substrate which is ready for transfer onto the substrate by wet transfer.

### Example 2: Mechanical Adhesion Testing

Mechanical adhesion strength of the product of example 1 was analyzed by tape testing. The tape tests demonstrated very stable anchoring of GO on the substrate. Anchoring was so stable that gold was detached from the titanium. This was surprising because the substrate is known for the good adhesion of the gold to the titanium. Detachment of gold from the titanium would not have been expected in the absence of covalent anchoring of GO.

### REFERENCE SIGNS

10 electrode
11 electrode carrier
12 electrode base material
14 carbon material
16 anchoring layer
18 anchoring structure
140 graphene material
160 monolayer

1. An electrode comprising an electrode base material, a carbon material, and an anchoring layer, wherein the anchoring layer is located between the electrode base material and the carbon material and comprises an anchoring structure having a chain of at least eight consecutive covalently bonded atoms.

2. The electrode according to claim 1,
wherein
the anchoring structure has a chain of at least 10 consecutive covalently bonded atoms.

3. The electrode according to claim 1,
wherein
the carbon material is a graphene material.

4. The electrode according to claim 1,
wherein
the carbon material is a graphene oxide or a reduced graphene oxide.

5. The electrode according to claim 1,
wherein
at least part of the anchoring layer is a monolayer.

6. The electrode according to claim 1,
wherein
a distal end of the anchoring structure is covalently bound to the carbon material.

7. The electrode according to claim 6,
wherein
the distal end of the anchoring structure is covalently bound to the carbon material by a distal substructure Ad-X or Ad=X
wherein Ad is a distal hetero atom of the anchoring structure, and
X is an atom of the carbon material.

8. The electrode according to claim 6,
wherein
the distal end of the anchoring structure is covalently bound to the carbon material by a distal substructure C(O)—NH—NH—X or C(O)—NH—N═X
wherein
C(O)—NH—NH or C(O)—NH—N is a subunit of the anchoring structure, and
X is an atom of the carbon material.

9. The electrode according to claim 1,
wherein
a proximal end of an anchoring structure is bound to the electrode base material.

10. The electrode according to claim 1,
wherein
the electrode base material comprises gold, iridium, platinum, titanium, silver, and/or palladium.

11. The electrode according to claim 10,
wherein
the gold, iridium, platinum, titanium, silver, and/or palladium is at a surface of the electrode base material.

12. The electrode according to claim 9,
wherein
a proximal end of the anchoring structure is coupled to the electrode base material by a proximal substructure
Ap-Y
wherein
Ap is a proximal hetero atom of the anchoring structure,
Y is an atom of the electrode base material, and
between Ap and Y designates a coupling of Ap to Y.

13. A method for preparing an electrode, which comprises the following steps:
a) contacting an electrode base material with an anchoring composition which comprises an anchoring compound having a chain of at least eight consecutive covalently bonded atoms,
b) contacting the electrode base material obtained from step a) with a carbon material.

14. An electrode carrier for detecting, receiving and/or inducing physiological electrical signals, wherein the electrode carrier comprises an electrode according to claim 1.

15. (canceled)
